(19) United States
(12) Patent Application Publication
(10) Pub. No.: US 2025/0378940 A1
(43) Pub. Date: Dec. 11, 2025

(54) Imaging Apparatus and Method for Determining an Imaging Protocol

(71) Applicant: Siemens Healthineers AG, Forchheim (DE)

(72) Inventors: Miriam Van de Stadt-Lagemaat, Erlangen (DE); Dominik Rupp, Tuchenbach (DE); Frank Mehler, Neunkirchen am Brand (DE)

(73) Assignee: Siemens Healthineers AG, Forchheim (DE)

(21) Appl. No.: 19/231,784

(22) Filed: Jun. 9, 2025

(30) Foreign Application Priority Data
Jun. 11, 2024 (EP) 24181398.9

(51) Int. Cl. G16H 30/40; G06T 7/00; G16H 30/20

(52) U.S. Cl. CPC G16H 30/40; G06T 7/0012; G16H 30/20; G06T 2200/24; G06T 2207/10088; G06T 2207/30036

(57) ABSTRACT

A computer-implemented method for determining an imaging protocol for the acquisition of image data of a body region of a patient using an imaging apparatus, the method comprising the steps of: acquiring information about the body region, performing a first imaging examination as a function of the information about the body region and acquiring image data of the body region, providing the image data and an input option for adjusting a parameter of an imaging region, providing an aid for adjusting the imaging region as a function of the information about the body region, acquiring an adjusted imaging region, and determining the imaging protocol for a second imaging examination as a function of the adjusted imaging region.

### BACKGROUND

Independent of the grammatical term usage, individuals with male, female or other gender identities are included within the term.

The planning of an imaging protocol for the acquisition of image data of a diagnostically relevant body region of a patient by means of an imaging apparatus can be dependent on patient-specific criteria and/or user-specific inputs during a patient registration as well as during a preparation of the patient for an imaging examination. For example, during the planning of the imaging protocol it is necessary to take account of a laterality of the diagnostically relevant body region (e.g., its presence in a left- or right-hand half of the body), a subdivision of the body region into relevant sections and/or an orientation of the body region in a reference system for the purpose of selecting a suitable slice orientation. In particular with imaging protocols for the tooth or jaw region of the patient, a specification of a suitable slice orientation as well as a selection of relevant sections can be complex due to the symmetrical structure of the dental arches (e.g., left, right or frontal lower and/or upper jaw) and the curved shape of the dental arches. That is why users of the imaging apparatus must undergo comprehensive training in order to ensure an error-free acquisition of image data of the diagnostically relevant body regions by means of an imaging apparatus in everyday clinical routine.

Commercially available imaging apparatuses are typically equipped with a prescribed user guide for the slice orientation at protocol level. However, such prescribed user guides seldom take account of the laterality of the diagnostically relevant body region and, for example, indicate a slice positioning for the left hip of a patient even though it is the right hip of the patient that is diagnostically relevant. Prescribed user guides should, in fact, be understood as examples, but the users of the imaging apparatus must possess a sufficient level of training and experience in order to plan measurements for all possible use cases as well as for complex anatomical structures.

In certain exceptional cases, when a small number of employed slice orientations are present, different parameters or scenarios are preset by default for the user, each slice orientation being stored with a corresponding user guide. However, this leads to a duplication of user guides, workflows, and imaging protocols, which is not desirable.

### SUMMARY

It is therefore an object of the disclosure to improve a process of determining an imaging protocol for an acquisition of image data of a body region of a patient by means of an imaging apparatus.

This object is achieved according to the disclosure by means of the subject matter of the independent claims. Advantageous aspects and beneficial developments are the subject matter of the dependent claims.

The computer-implemented method according to the disclosure for determining an imaging protocol for the acquisition of image data of a body region of a patient by means of an imaging apparatus comprises the following steps:
acquiring information about the body region,
performing a first imaging examination as a function of the information about the body region and acquiring image data of the body region,
providing the image data and an input option for adjusting a parameter of an imaging region,
providing an aid for adjusting the imaging region as a function of the information about the body region,
acquiring an adjusted imaging region, and
determining the imaging protocol as a function of the adjusted imaging region.

An imaging apparatus can constitute a device which is designed to acquire image data of an examination subject, in particular of a body region or an interior of a patient. Preferably, an imaging apparatus is designed to record two-dimensional and/or three-dimensional image data, in particular time-dependent three-dimensional image data, of the examination subject. Examples of imaging apparatuses include magnetic resonance devices, X-ray machines, computed tomography devices, single-photon emission computed tomography systems, positron emission tomography systems, as well as mammography devices, ultrasound scanners, and the like. In a preferred aspect, the imaging apparatus is embodied as a magnetic resonance device.

An imaging protocol for an imaging examination can be characterized by one or more imaging parameters. Examples of imaging parameters include a spatial resolution, a contrast, a slice thickness, a dimension of an imaging volume, a relaxation time, an echo time, and the like. An imaging parameter can comprise an arbitrary image-relevant setting of the imaging apparatus, but also a parameter of a workflow of the imaging examination. Furthermore, parameters that define an imaging region may also be understood as imaging parameters.

An imaging examination may comprise one or more groups of imaging parameters as well as one or more imaging sequences.

An imaging protocol can define a workflow of an imaging examination of a body region of a patient. For example, the performance of an imaging examination may comprise performing one or more imaging sequences. Image data of the body region of the patient can be acquired when the imaging sequences are performed.

The imaging protocol can be adjusted to acquire image data of a body region. The body region may, for example, comprise a hip region, a shoulder region, a knee region, a brain region, an eye region, or a random other anatomical structure. It is equally conceivable that the body region comprises a tissue structure or an organ, such as, e.g., a heart, a liver, a kidney, or the like.

Preferably, the imaging protocol or an imaging examination based on the imaging protocol comprises an execution of one or more imaging sequences which are adjusted for imaging of a jaw region, a tooth region, and/or a tooth.

In a preferred aspect, the first imaging examination and/or the second imaging examination are magnetic resonance examinations of a jaw region or a tooth region of a patient. It is conceivable that an imaging sequence of at least one magnetic resonance examination has a very short echo time in order to compensate for a short T2 relaxation time of spins of a dentine or a tooth enamel of a tooth and to visualize these regions at high signal intensity in acquired image data. Very short echo times can be less than 150 μs or less than 70 μs. FLASH (Fast Low-Angle Shot) or UTE (Ultra-short Echo Time) sequences represent examples of possible imaging sequences. However, it is also conceivable to make use of imaging sequences with a longer echo time, such as, e.g., a TSE (Turbo Spin Echo) sequence. With such sequences, an acquisition of the magnetic resonance signal of the tooth enamel or the dentine can be avoided. In image data of such imaging sequences, the teeth can be differentiated based on a lack of signal intensity in comparison with a surrounding tissue.

Image data can constitute any data that is acquired from the body region of the patient by means of the imaging apparatus. Image data can comprise both raw data and images that are derived from the raw data. For example, the image data may comprise digitized magnetic resonance signals acquired by a magnetic resonance device. The image data can be stored as complex values in a k-space matrix. Preferably, however, the image data also comprises magnetic resonance images which have been reconstructed as a function of the digitized magnetic resonance signals.

Information about the body region of a patient can comprise any description of a type, a designation, a position, and/or a dimension of the body region of the patient. The information about the body region can further comprise a selection and/or an identification of a body region or an anatomical structure from a list or a database of body regions.

The acquisition of the information about the body region of the patient preferably comprises a registration of an input by a user of the imaging apparatus by means of a suitable input interface or user interface, such as e.g., a mouse, a keyboard, a touchscreen, and/or a conversational interface. The acquisition of the information about the body region of the patient may further comprise a retrieval or receiving of data, in particular of patient data or patient information, from an internal or external memory unit and/or from a medical information system by means of an interface. A radiological information system (RIS) or a hospital information system (HIS) may be a representative example of a medical information system.

The information about the body region of the patient can be input manually by a user of the imaging apparatus by means of a user interface. In particular, the information about the body region can be selected by the user of the imaging apparatus by means of a selection of an anatomical structure or a section of an anatomical structure as a function of a representation of the anatomical structure provided by means of the user interface. However, it is equally conceivable that the imaging apparatus comprises a control unit and/or a computing unit which are designed to obtain the information about the body region of the patient as a function of patient information by means of a suitable interface from the medical information system, from a cloud and/or a local memory unit. In particular, the information about the body region can be determined by means of an algorithm or an image processing algorithm as a function of the image data of the first imaging examination and/or of the patient information. The patient information may comprise a medical report or a diagnosis, as well as an age, a gender, a weight, or any other medical or demographic information about the patient.

In a preferred aspect of the method according to the disclosure, the acquisition of the information about the body region of the patient comprises an acquisition of a section of a tooth region of the patient, in particular, a section of one or more dental arches.

The first imaging examination is preferably a localizer measurement. A localizer measurement may be understood as a time-efficient imaging examination in which image data, in particular localizer image data, of the body region of the patient is acquired. The localizer measurement may exhibit limitations with respect to a quality and/or a spatial resolution of the acquired image data compared to a conventional imaging examination. The localizer measurement preferably provides a spatial resolution which is suitable for detecting and/or identifying anatomical structures, such as, e.g., a tooth, a dental arch, a jawbone, or the like. The first imaging examination may further comprise a projection measurement. A projection measurement can constitute an imaging examination in which a spatial encoding is omitted in one spatial direction. The image data can therefore comprise a two-dimensional projection image of a three-dimensional volume of the body region of the patient. It is further conceivable that the image data comprises an image from a previous imaging examination, in particular, a previous magnetic resonance examination.

In a preferred aspect of the method according to the disclosure, image data of the body region is captured by means of an antenna element or a plurality of antenna elements of a magnetic resonance device. For this purpose, the antenna elements can be positioned, for example, on a jaw region and/or in an oral cavity of the patient. The antenna element or the plurality of antenna elements can be configured in particular to receive magnetic resonance signals of the jaw region and to transmit these signals to a receive unit of the magnetic resonance device.

The first imaging examination can be performed as a function of the information about the body region. However, it is also conceivable for the first imaging examination to be performed independently of the acquisition of the information about the body region of the patient (e.g., as an independent localizer measurement or survey scan). The performance of the first imaging examination permits image data of the body region of the patient to be acquired. The image data may in particular comprise localizer image data or further image data according to a below-described aspect.

Providing the image data of the body region of the patient preferably comprises at least storing the image data on a local memory unit of the imaging apparatus, on a network storage unit, in a medical information system, and/or in a cloud. It is furthermore conceivable that the providing of the image data of the body region comprises an outputting of the image data to a user of the imaging apparatus by means of an output unit. An output unit can be embodied, for example, as a screen, a monitor, a touchscreen, a projector, or the like.

A user of the imaging apparatus may be, for example, a medical practitioner, in particular a dental specialist, a medical technical assistant, or a member of the medical staff of a practice or a clinical institution. The user may be situated at an installation site of the imaging apparatus, but also at any other location. For example, the user may be stationed in another town, another region, and/or another country and interact by remote control with the imaging apparatus.

It is furthermore conceivable for the image data of the body region of the patient to be processed by a program and/or an algorithm which are/is designed to categorize, to identify, and/or to classify the contents of an image or one or more anatomical structures. It is conceivable, in particular, that the program and/or the algorithm are/is designed to determine an aid for adjusting the imaging region and/or a provisional imaging region as a function of the information about the body region and the image data. In one aspect of the method according to the disclosure, the program and/or the algorithm are/is designed to determine the aid for adjusting the imaging region and/or the provisional imaging region as a function of patient information.

Providing the input option for adjusting the parameter of an imaging region preferably comprises an outputting of the input option by means of an output unit and/or a user interface. For example, the input option for adjusting the parameter of the imaging region can be output to the user by means of a graphical user interface, in particular, a monitor or a touchscreen. The input option for adjusting the parameter of the imaging region can comprise an input mask that allows the user to change or adjust a parameter of the imaging region. A parameter of the imaging region can, for example, define a spatial position, an orientation, a dimension, and/or a shape of the imaging region.

The imaging region may be understood as a measurement volume, a field of view, a viewing window, or a slice orientation. In particular, the imaging region can define a volume within a patient receiving zone of the imaging apparatus from which image data is acquired by means of an imaging examination. Preferably, at least one section of the body region of the patient is positioned for an imaging examination in a space called an imaging volume of the imaging apparatus. The imaging region may be understood as a volume within the imaging volume of the imaging apparatus to which the acquisition of image data is limited.

In a preferred aspect, the imaging apparatus is embodied as a magnetic resonance device. An imaging volume may constitute a volume having a maximum homogeneity of a magnetic field, in particular, an isocenter, of the magnetic resonance device.

Providing an aid for adjusting the imaging region preferably comprises outputting information relating to a desired or ideal setting of the imaging region by means of an output unit or a graphical user interface according to an above-described aspect. For example, the aid for adjusting the imaging region can be displayed for the user on a monitor.

The aid for adjusting the imaging region is provided as a function of the information about the body region.

Providing the aid for adjusting the imaging region may comprise selecting information relating to a desired or ideal setting of the imaging region as a function of the information about the body region of the patient. For example, a control unit and/or a computing unit of the imaging apparatus can be designed to select and provide a specific piece of information relating to a desired or ideal setting of the imaging region from a library or a database as a function of the body region of the patient. It is conceivable that the information about the body region of the patient comprises a name, a designation, and/or an identification of an anatomical structure to which reference is made by means of the control unit and/or the computing unit when determining the aid for adjusting the imaging region.

In one example, the information about the body region of the patient comprises a designation of a section of an anatomical structure, in particular, a section of a tooth region or a dental arch. The aid for adjusting the imaging region can be selected accordingly as a function of the designation of the section of the anatomical structure and provided to the user of the imaging apparatus by means of an output unit or a graphical user interface. For example, the aid for adjusting the imaging region can be selected and retrieved from a database of a memory unit, which comprises a plurality of aids for adjusting the imaging region for different body regions.

The aid for adjusting the imaging region can be designed to inform a user of the imaging apparatus about a desired or ideal dimension, position, and/or orientation of the imaging region for the body region of the patient. The aid for adjusting the imaging region can further be designed to support the user of the imaging apparatus during an adjustment of the dimension, position, and/or orientation of the imaging region relative to an anatomical structure of the body region of the patient. It is conceivable that the aid for adjusting the imaging region represents a guideline or instruction that specifies how the imaging region should be parameterized for an imaging examination of the body region of the patient or of a section of the body region of the patient. An imaging region parameterized according to the aid for adjusting the imaging region can be aligned along a diagnostically relevant anatomical structure of the body region of the patient and/or adjusted to fit the diagnostically relevant anatomical structure of the body region of the patient.

In a preferred aspect of the method according to the disclosure, providing the aid for adjusting the imaging region comprises overlaying and/or juxtaposing the aid for adjusting the imaging region with the image data of the first imaging examination. For example, the aid for adjusting the imaging region and the image data of the first imaging examination can be provided to a user of the imaging apparatus by means of an output unit or a graphical user interface.

An acquisition of an adjusted imaging region can comprise an acquisition of an input by a user of the imaging apparatus. In particular, the input by the user can be made by means of an input interface or user interface according to an above-described aspect. The input of the user can comprise an adjusting of a parameter or a property of a graphical object which represents the imaging region. In particular, the input can represent an adjustment of a position, a dimension, and/or an orientation of the graphical object. The graphical object can comprise a window or any desired polygon, in particular a square, a rectangle, or some other multisided shape. The graphical object is preferably superimposed as an overlay on the image data of the first imaging examination. The graphical object can, in particular, match the input option for adjusting the parameter of an imaging region.

It is further conceivable that the input by the user comprises a text-based input. The text-based input can comprise one or more coordinates, one or more dimensions of the imaging region, a coordinate of a geometric midpoint of the imaging region, a rotation angle of the imaging region, or the like.

The method according to the disclosure preferably enables the imaging region to be adjusted by a user of the imaging apparatus as a function of the aid for adjusting the imaging region and the image data of the first imaging examination. This advantageously enables the imaging region to be adjusted taking into account both rules or specifications for setting the imaging region for specific body regions and individual preconditions of an anatomical structure of the body region of the patient.

Determining the imaging protocol for a second imaging examination can comprise determining one or more imaging parameters for the second imaging examination. It is equally conceivable that determining the imaging protocol comprises determining one or more imaging sequences, in particular, a succession of multiple imaging sequences. The imaging protocol can determine one or more imaging parameters, but also a workflow, of the second imaging examination or of further imaging examinations. The second imaging examination, as well as the further imaging examinations, can be designed to acquire high-resolution image data of the body region of the patient.

According to the disclosure, the imaging protocol is determined as a function of the adjusted imaging region. Preferably, the imaging protocol is determined automatically as a function of the imaging region adjusted by the user. For example, one or more groups of imaging parameters, one or more imaging sequences, and/or a succession of imaging sequences can be determined as a function of the adjusted imaging region (e.g., based on the input by the user). The imaging protocol is preferably determined as a function of the adjusted imaging region by means of an algorithm implemented on a control unit and/or a computing unit of the imaging apparatus.

An algorithm mentioned herein can comprise a logic-based algorithm, a trained algorithm, a self-learning algorithm, an artificial neural network, a machine learning algorithm, an image processing algorithm, as well as one or more mathematical operators.

Certain body regions of patients may have significant differences from one another individually. For example, a size and/or gender of a patient, but also a girth, can affect a position of certain body regions. However, it is also conceivable that anatomical structures of the body region of the patient have different shapes, dimensions, or spatial orientations. Furthermore, anatomical structures that are present in both halves of the body of the patient are often not completely symmetrical to one another and can confuse or irritate a user when determining an imaging protocol. In particular, anatomical structures that can be subdivided into several approximately similar or symmetrical diagnostically relevant regions require an intensive training of users of the imaging apparatus in order to ensure that image data of the correct body region is acquired and that the acquired image data is of high quality.

A method according to the disclosure enables a guided instruction of a user when determining an imaging protocol for the acquisition of image data of a body region of a patient by means of an imaging apparatus. In particular, the method according to the disclosure can support the user dynamically, i.e., as a function of different diagnostically relevant body regions of the patient and/or as a function of image data of a specific body region of a patient, in the adjustment of an imaging region. For example, as a result of the aid for adjusting the imaging region, the user can be pointed directly to the diagnostically relevant body region and a parameterization of the imaging region consistent with standard practice without specifying or limiting the imaging region in advance. This can be advantageous, in particular, in an imaging examination of teeth, which can yield very different results between patients and can easily lead to confusion due to the symmetry between different tooth sections.

Furthermore, the method according to the disclosure can enable a user of an imaging apparatus to adequately determine an imaging region without special prior knowledge, even in the presence of complex anatomical structures and/or slice orientations. Errors in the determination of an imaging protocol and in a subsequent acquisition of further image data of the body region can advantageously be avoided as a result. Moreover, a high quality of the further image data acquired by means of the imaging apparatus can be ensured.

By means of the method according to the disclosure, the image data of the body region, the input option for adjusting the parameter of the imaging region, and the aid for adjusting the imaging region can be provided to the user of the imaging apparatus simultaneously or in a predetermined order. This can advantageously enable the user to adjust the imaging region to fit one or more sections of the body region of the patient in a more efficient and/or more precise manner. In particular, the user can be supported within the framework of a guided human-machine interaction as a function of the image data of the body region, the input option for adjusting the parameter of the imaging region and the aid for adjusting the imaging region by means of a user interface of the imaging apparatus when adjusting the imaging region to fit one or more sections of the body region of the patient.

In one aspect, the method according to the disclosure comprises the step:
determining a provisional imaging region as a function of the image data,
wherein providing the input option for adjusting the parameter of the imaging region comprises an overlaying of a representation of the provisional imaging region with the image data of the body region of the patient.

Determining the provisional imaging region can, in particular, comprise determining a dimension, a spatial arrangement, and/or a spatial orientation of the imaging region relative to the body region of the patient.

When the image data is provided, the image data of the body region of the patient is preferably superimposed as an overlay on the representation of the provisional imaging region. A representation of the provisional imaging region can, for example, comprise a symbol, a graphical object, or a graphical visualization of the provisional imaging region. In particular, the representation of the provisional imaging region can comprise a window or a polygon according to an above-described aspect. Providing an overlay of the provisional imaging region with the image data of the first imaging examination can provide a user of the imaging apparatus with information relating to a preferred or conventional dimension, orientation, and/or spatial position of the provisional imaging region in relation to the body region of the patient.

In a preferred aspect, the image data of the body region of the patient is superimposed as an overlay on the representation of the provisional imaging region and provided together with the input option for adjusting the parameter of the imaging region. This allows a starting point to be provided for adjusting the imaging region by the user and an efficiency of an adjustment of the imaging region to be advantageously increased.

It is conceivable that the provisional imaging region is determined as a function of the image data by means of an algorithm, in particular, an image processing algorithm. For example, the algorithm can be designed to process the image data or localizer image data and to determine a dimension, a spatial arrangement, and/or an orientation of the provisional imaging region relative to the body region of the patient. It is further conceivable that the provisional imaging region is determined as a function of a specification or guideline for parameterizing an imaging region for a specific body region, a reference imaging examination of the same body region of a different patient, and/or a geometric analysis of the body region of the patient.

Providing an aid for adjusting the imaging region and a representation of a provisional imaging region superimposed on the image data can enable or make it easier for inexperienced users, in particular, to adjust the imaging region in the case of complex or atypically formed anatomical structures, but also in the case of difficult slice orientations.

Furthermore, providing the aid for adjusting the imaging region and the provisional imaging region enables the user to make a dynamic assessment of any differences between the aid for adjusting the imaging region and the provisional imaging region, which may occur, e.g., due to complex or atypically shaped anatomical structures. As a result, the user can advantageously be alerted to problems in the adjusting of the imaging region, and a risk of an erroneous determination of the imaging protocol can be reduced.

In a further aspect of the method according to the disclosure, the acquisition of the adjusted imaging region comprises an acquisition of a correction of the provisional imaging region.

The acquisition of the adjusted imaging region preferably comprises an acquisition of a correction of a provisional dimension, a provisional spatial arrangement, and/or a provisional orientation of the imaging region relative to the body region of the patient.

In particular, providing the input option for adjusting the parameter of the imaging region can comprise a prompt requesting the user of the imaging apparatus to make an adjustment or a correction of the provisional imaging region. The input option for adjusting the parameter of the imaging region can be provided to the user by means of an output unit and/or a graphical user interface according to an above-described aspect.

Diagnostically relevant body regions having small dimensions, such as, e.g., an inflammation of a pulp or a tooth root, must typically be taken into account when determining an imaging protocol and cannot simply be covered by means of conventional methods for determining the imaging region. With anatomical structures whose dimensions and/or orientation diverge from a typical norm or standard distribution, the process of determining the imaging region acquires additional difficulty.

A method according to the disclosure enables a provisional imaging region to be corrected with the help of a provided aid for adjusting the imaging region. In particular, a direct comparison of the image data of the body region of the patient with the provisional imaging region and the aid for adjusting the imaging region advantageously enables the user to make a dynamic assessment of measures required for the individual correction or adjustment of the imaging region for a specific anatomical structure of a patient. As a result, a correct assignment of the imaging region can also be ensured in the case of anatomical structures that deviate from the norm.

In a preferred aspect, the method according to the disclosure comprises the further step:

performing the second imaging examination for the acquisition of further image data of the body region of the patient as a function of the imaging protocol.

The further image data can differ from the image data of the first imaging examination in particular by a higher quality, an improved centering on a diagnostically relevant anatomical structure and/or an increased spatial resolution.

It is conceivable that the first imaging examination comprises a localizer measurement, while the second imaging examination comprises a high-resolution imaging examination focused on the body region or a section of the body region of the patient. The localizer measurement can, in particular, fulfill the function of enabling the user to adjust a parameter of the imaging protocol for the second imaging examination by providing the image data containing the specific shape of the body region of the patient.

The first imaging examination and the second imaging examination are preferably performed by means of the same imaging apparatus. However, it is also conceivable that the first imaging examination and the second imaging examination are performed by means of different imaging apparatuses (e.g., different magnetic resonance devices) or different imaging modalities (e.g., an X-ray machine and a magnetic resonance device). For this purpose, the patient can be positioned, for example, by means of a stereotactic patient receiving device in a reproducible position relative to the first imaging apparatus and the second imaging apparatus. Furthermore, markers can be used in order to facilitate an adjustment of the imaging region for determining the imaging protocol for the second imaging examination by means of the second imaging apparatus. The first imaging examination and the second imaging examination can thus be performed at different times and/or at different locations.

Performing a second imaging examination as a function of an imaging protocol determined on the basis of a first imaging examination enables the second imaging examination to be adjusted to match individual preconditions of a body region of a patient. In particular, the method according to the disclosure permits a reproducible acquisition of further image data of the body region with high quality, irrespective of the experience of the user of the imaging apparatus.

In an aspect of the method according to the disclosure, providing the aid for adjusting the imaging region comprises an outputting of a text-based user guide and/or a graphical user guide.

A text-based user guide can comprise a description or an instruction which contains information relating to a desired or ideal parameter of the imaging region for a specific imaging protocol, a specific body region and/or a specific section of a body region. In particular, the text-based user guide can contain a suggestion or a guideline for adjusting a parameter of the imaging region and/or of a procedure for adjusting a parameter of the imaging region as a function of a body region that is to be examined and/or a section of a body region that is to be examined. For example, the text-based user guide can comprise a description of a desired or ideal parameter of the imaging region for a specific section of an anatomical structure of the patient, in particular a section of a dental arch or sections of both dental arches.

A parameter of the imaging region can define a dimension, a spatial position, and/or an orientation of the imaging region relative to the body region of the patient. A parameter of the imaging region can, in particular, represent an imaging parameter of an imaging protocol.

A graphical user guide can comprise pictographic or pictorial information relating to a desired or ideal parameter of the imaging region for a specific imaging protocol, a specific body region, and/or a specific section of a body region. Preferably, the graphical user guide comprises a desired or ideal dimension, a desired or ideal spatial position, and/or a desired or ideal orientation of the imaging region relative to the body region of the patient.

Both the text-based user guide and the graphical user guide can point a user of the imaging apparatus to a desired or ideal parameter of the imaging region as a function of the body region of the patient. The user can, however, decide, as a function of the individual anatomical preconditions of the body region of the patient (e.g., as a function of the image data of the first imaging examination), to depart from the desired or ideal parameter of the imaging region when adjusting the imaging region.

By providing a text-based and/or graphical user guide, it is also possible for inexperienced users to be informed about a parameterization of the imaging region consistent with standard practice as a function of the body region. Costs for training staff, as well as errors in determining the imaging protocol, can be advantageously reduced as a result.

In a preferred aspect of the method according to the disclosure, providing the aid for adjusting the imaging region comprises the outputting of a graphical user guide, the graphical user guide comprising a representation of the body region and of the imaging region.

The graphical user guide can, in particular, comprise a detailed or abstract graphical representation of the imaging region in a desired position and/or orientation relative to the body region of the patient. Preferably, the graphical user guide further comprises a representation of the body region of the patient in a correct spatial arrangement relative to the abstract graphical representation of the imaging region. The graphical user guide may further comprise a symbol and/or a sign that informs the user about a change in a parameter of the imaging region in order to achieve a desired or ideal relative arrangement of the imaging region with respect to the body region of the patient. For example, the graphical user guide may contain an arrow and/or a line that points the user to a change in the orientation, size, and/or position of the imaging region.

A graphical user guide permits the imaging region to be adjusted by means of a direct visual juxtaposition of an arrangement and/or a shape of the representation of the imaging region with the image data of the first imaging examination and/or a provisional imaging region according to an above-described aspect. This advantageously enables a process of adjusting the imaging parameter to be simplified or completed faster. Furthermore, a use of technical terms which may be necessary when using a text-based user guide can advantageously be avoided. The adjustment of the imaging region can advantageously be accomplished also by inexperienced users as a result.

A further advantage of a graphical user guide with a representation of the body region of the patient consists in the possibility to juxtapose the ideal or desired imaging region relative to the body region of the patient with the image data of the body region of the patient, but also with the provisional imaging region. The graphical user guide can advantageously reveal problems in the adjustment of the imaging region to fit an anatomical structure of the patient, which remain hidden from the user in a text-based or a parameter-based adjustment of the imaging region.

In a preferred aspect of the method according to the disclosure, the graphical user guide comprises at least two sectional views of the body region of the patient. The at least two sectional views of the body region of the patient are aligned along different reference planes that are preferably orthogonal to one another.

For example, a reference plane of a first sectional view of the at least two sectional views of the body region is arranged parallel to a frontal plane of the patient. A reference plane of a second sectional view of the at least two sectional views of the body region can be arranged parallel to a sagittal plane of the patient. It is also conceivable that the graphical user guide comprises a third sectional view of the body region of the patient. A reference plane of the third sectional view of the body region can be aligned parallel to a transverse plane of the patient. The graphical user guide can, of course, also comprise just one sectional view or just two sectional views of the body region, the reference planes of which are in each case arranged parallel to the sagittal plane, the frontal plane, or the transverse plane of the patient.

Preferably, the reference planes of the at least two sectional views of the body region are aligned substantially orthogonally to one another. This can mean that an orientation of the reference planes of the first sectional view and of the second sectional view diverge by a few degrees, for example, less than 10°, less than 8°, less than 6° or less than 4°, from a right-angled orientation.

It is further conceivable that a coordinate system defined by the reference planes of the at least two sectional views of the body region is inclined by an angle with respect to a coordinate system which is defined by the sagittal plane, the frontal plane and the transverse plane of the patient.

Providing a graphical user guide according to the disclosure permits a visualization of a desired or ideal arrangement of the imaging region in at least two reference planes. This advantageously enables a user of the imaging apparatus to determine a difference between a current arrangement of the imaging region and/or a provisional imaging region and the desired or ideal arrangement of the imaging region with regard to the reference planes of the at least two sectional views while taking account of an individual shape of the body region of the patient. In particular, providing multiple sectional views of the body region can allow the user of the imaging apparatus to perform a more precise adjustment of the imaging region to fit individual shapes of anatomical structures, such as, e.g., a dental arch of the patient.

In a further aspect of the method according to the disclosure, providing the image data and the input option for adjusting the parameter of the imaging region comprises an outputting of at least two sectional views of the body region of the patient based on the image data, wherein a reference plane of each of the at least two sectional views of the body region of the patient based on the image data is aligned parallel to a reference plane of each of the at least two sectional views of the body region of the patient of the graphical user guide.

For example, a reference plane of a first sectional view of the body region based on the image data is aligned substantially parallel to the reference plane of the first sectional view of the body region of the graphical user guide. Similarly, a reference plane of a second sectional view of the body region based on the image data can be aligned substantially parallel to the reference plane of the second sectional view of the body region of the graphical user guide.

In a preferred aspect, providing the image data and the input option for adjusting the parameter of the imaging region comprises an outputting of a third sectional view of the body region based on the image data, wherein a reference plane of the third sectional view of the body region based on the image data is aligned parallel to a reference plane of a third sectional view of the body region of the graphical user guide.

In a particularly preferred aspect, the reference plane of the first sectional view of the body region based on the image data corresponds to the reference plane of the first sectional view of the body region of the graphical user guide. Similarly, the reference planes of the second and/or third sectional views of the body region based on the image data can correspond to the respective reference planes of the second and/or third sectional view of the body region of the graphical user guide.

A sectional view of the body region based on the image data can represent a two-dimensional image, for example, a magnetic resonance image, an X-ray image, or a computed tomography image, of the body region of the patient. It is conceivable that the sectional view of the body region based on the image data comprises a transverse sectional image, a frontal sectional image, and/or a sagittal sectional image of the body region. The sectional view of the body region based on the image data may in particular comprise a transverse sectional image, a frontal sectional image and/or a sagittal sectional image of a localizer measurement of the body region of the patient.

A graphical user guide containing a representation of the body region of the patient can differ significantly from an individual shape and/or arrangement of the body region in a patient. By providing a graphical user guide containing sectional views of the body region and, corresponding thereto, sectional views of the body region based on the image data it is possible to approximate an arrangement and/or orientation of the imaging region in several spatial directions to a desired or ideal result. Furthermore, individual preconditions of a shape and/or an orientation of the body region and/or of a section of the body region of the patient can simultaneously be taken into account when adjusting the imaging region. As a result, an efficiency of the adjustment of the imaging region can advantageously be increased and/or an amount of time required by a user of the imaging apparatus to prepare or parameterize the second imaging examination can be reduced.

In a preferred aspect of the method according to the disclosure, providing the image data and the input option for adjusting the parameter of the imaging region comprises juxtaposing the image data with the graphical user guide.

Preferably, the graphical user guide comprises a sectional view of the body region of the patient according to an above-described aspect. Similarly, the image data can comprise a sectional view of the body region of the patient. According to an above-described aspect, the reference planes of the sectional view of the body region of the graphical user guide and the sectional view of the body region based on the image data can be aligned parallel to one another or correspond to one another.

It is conceivable that the image data together with the graphical user guide is provided to the user of the imaging apparatus by means of an output unit or a graphical user interface. The image data and the graphical user guide can in this case be shown arranged side by side or at least partly overlapping. Preferably, the image data and the graphical user guide are provided by means of the output unit or the graphical user interface in such a way that the image data and the graphical user guide are presented together on a display area visible to the user at a specific point in time.

A juxtaposition of the image data and the graphical user guide enables a visual adjustment of the imaging region to fit an individual anatomical structure of the body region of the patient while at the same time taking into account a desired or ideal arrangement and/or orientation of the imaging region to a standard case visualized by means of the graphical user guide. This advantageously enables confusion in the selection of sections of the body region, but also errors in the adjustment of the imaging region, to be reduced or avoided.

A desired or ideal arrangement and/or orientation of the imaging region with respect to a specific body region can be defined by a specification or a guideline of an expert, a facility, an institution and/or a user panel.

In a further aspect of the method according to the disclosure, the body region comprises an anatomical region of a first half of the body of the patient, which is formed substantially symmetrically to an anatomical region in a second half of the body of the patient opposite the first half of the body of the patient.

Preferably, the first half of the body of the patient is formed substantially symmetrically to the second half of the body of the patient. It is conceivable that the first half of the body and the second half of the body of the patient face each other symmetrically at a median plane of the patient.

For example, the body region of the patient may comprise a part of an extremity, such as, e.g., an arm, a hand, a foot, a knee, a shoulder, or the like. The body region may further comprise a joint, such as e.g., a knee joint, a shoulder joint, a hip joint, a wrist, but also a part of an organ, such as e.g., a cerebral hemisphere, a half of the lung, or the like.

In a preferred aspect of the method according to the disclosure, the body region of the patient comprises a jaw region and/or a tooth region. For example, the jaw region and/or the tooth region comprise a set of teeth, a section of a jawbone, a section of a set of teeth, one or more dental arches, a section of a dental arch, a gum, a section of a gum and/or one or more teeth of the patient.

A tooth region of a patient comprises two substantially uniform dental arches, which in turn have sections on the left half of the body of the patient and the right half of the body of the patient. Also, rows of front teeth of the dental arches are arranged at an angle to rows of back teeth of the dental arches; for which reason, numerous sections of the tooth region need to be differentiated in the imaging examination of the tooth region. The image data of such sections of the tooth region can be very similar optically, as a result of which a risk of confusing a right side with a left side of a dental arch or a section of a lower dental arch with a section of an upper dental arch is increased.

By providing the aid for adjusting the imaging region as a function of the information about the body region, it is possible to establish a direct reference to a section of the body region relevant to the further imaging examination. By this means, a risk of adjusting the imaging region for a wrong section of the body region by a user of the imaging apparatus can advantageously be reduced or avoided even in the case of complex anatomical structures having different sections.

In a preferred aspect of the method according to the disclosure, a reference plane of a first sectional view of the at least two sectional views of the body region of the patient is aligned parallel to an occlusal plane of the patient along a section of a dental arch. A reference plane of a second sectional view of the at least two sectional views of the body region of the patient is aligned orthogonally to the first sectional view of the body region of the patient.

As described above, a coordinate system defined by the reference planes of the at least two sectional views of the body region are inclined by an angle with respect to a coordinate system which is defined by the sagittal plane, the frontal plane and the transverse plane of the patient. The angle of the inclination can, in particular, correspond to an angle between a plane defined by the occlusal plane of the patient and a transverse plane of the patient.

Providing a sectional view of the body region aligned parallel to an occlusal plane of the patient along a section of a dental arch enables an orientation of the imaging region relative to a curvature or trajectory of a dental arch, which can be different with different patients, in particular patients of different ages, to be taken into account. As a result, an adjustment of the imaging region along a three-dimensional extension of a diagnostically relevant section of the body region of the patient by a user can advantageously be simplified, and/or a risk of errors occurring during the adjustment of the imaging region can be reduced.

The imaging apparatus according to the disclosure is designed to acquire image data of a body region of a patient. The imaging apparatus can be constructed according to an above-described aspect.

In a preferred aspect, the imaging apparatus is implemented as a magnetic resonance device. The magnetic resonance device can be embodied to acquire magnetic resonance data, in particular magnetic resonance images, of a patient positioned in a patient receiving zone of the magnetic resonance device. The magnetic resonance data or magnetic resonance image data can comprise image data, in particular localizer image data, according to an above-described aspect.

The magnetic resonance device is designed to perform a first imaging examination, but also a second imaging examination, of the body region of the patient according to an above-described aspect of the method according to the disclosure. By using a magnetic resonance device, it is advantageously possible to avoid exposing the patient to ionizing radiation by comparison with X-ray machines or computed tomography devices.

The imaging apparatus comprises a control unit, an output unit, and a user interface. According to the disclosure, the control unit is designed to coordinate a method as claimed in one of the preceding claims and to perform said method by means of the imaging apparatus. The control unit can be integrated in the imaging apparatus or be implemented as a standalone component.

The output unit is designed to provide a user of the imaging apparatus with the aid for adjusting the imaging region. The user interface is designed to enable the user to adjust the imaging region as a function of the aid for adjusting the imaging region.

The output unit and the user interface can be designed according to an above-described aspect. It is conceivable that the output unit and the user interface are integrated in one component. However, the output unit and the user interface can also be implemented as autonomous components. In a preferred aspect, the output unit and the user interface form a graphical user interface which is designed to provide the user of the imaging apparatus with the aid for adjusting the imaging region and to allow the user to adjust the imaging region.

Preferably, the control unit has a signal connection to the user interface and/or the output unit of the imaging apparatus. In particular, the control unit can be designed to actuate the output unit to output the aid for adjusting the imaging region to the users of the imaging apparatus. The control unit can be further designed to register adjustments of the imaging region undertaken by the user by means of the user interface.

The imaging apparatus according to the disclosure shares the advantages of a method according to an above-described aspect.

The components of the imaging apparatus according to the disclosure can advantageously be coordinated with one another so as to enable a time-efficient and robust performance of a method according to the disclosure. In particular, the imaging apparatus according to the disclosure can be designed to coordinate and perform a workflow sequence of individual method steps independently. The user of the imaging apparatus can thus be enabled to adjust the imaging region for a further imaging examination of the body region of the patient without the requirement for the user to have particular specialized knowledge or to undergo exhaustive training.

The computer program product according to the disclosure can be loaded directly into a memory unit of a computing unit of an imaging apparatus according to the disclosure. The computer program product comprises program code means for performing a method according to an above-described aspect when the computer program product is executed in the computing unit of the imaging apparatus.

The computer program product according to the disclosure enables the method according to the disclosure to be performed quickly, identically, reproducibly, and robustly. The computer program product is configured such that it can perform the method steps according to the disclosure by means of the computing unit. Preferably, the computing unit comprises necessary prerequisites, such as, for example, a main memory, a graphics card, or a logic unit, so that the respective method steps can be performed efficiently. The computer program product is stored, for example, on a computer-readable medium or on a network, a server, or in a cloud, from where it can be loaded into a processor of the computing unit. The computing unit can, in this case, be designed as a self-contained system component or as a part of the imaging apparatus. Furthermore, control information of the computer program product can be stored on an electronically readable data medium. The control information of the electronically readable data medium can be embodied such that when the data medium is used in the computing unit of the imaging apparatus, it performs a method according to the disclosure. Examples of electronically readable data media are a DVD, a magnetic tape, a USB stick, or any other data storage device on which electronically readable control information, in particular software, is stored. When this control information is read from the data medium and transferred to a control unit and/or to the computing unit of the imaging apparatus, all the aspects of the described method according to the disclosure can be performed.

### BRIEF DESCRIPTION OF THE DRAWINGS

Further advantages and specific details of the present disclosure will become apparent from the exemplary aspects described in the following, as well as with reference to the drawings, in which:

FIG. 1 shows a schematic representation of an aspect of an imaging apparatus according to the disclosure,

FIG. 2 shows a flowchart of an aspect of a method according to the disclosure,

FIG. 3 shows a schematic representation of an aid for adjusting the imaging region of an aspect of a method according to the disclosure,

FIG. 4 shows a schematic representation of an aid for adjusting the imaging region of an aspect of a method according to the disclosure,

FIG. 5 shows a schematic representation of an aid for adjusting the imaging region of an aspect of a method according to the disclosure,

FIG. 6 shows a schematic representation of an aid for adjusting the imaging region of an aspect of a method according to the disclosure, and

FIG. 7 shows a schematic representation of an aid for adjusting the imaging region of an aspect of a method according to the disclosure.

### DETAILED DESCRIPTION

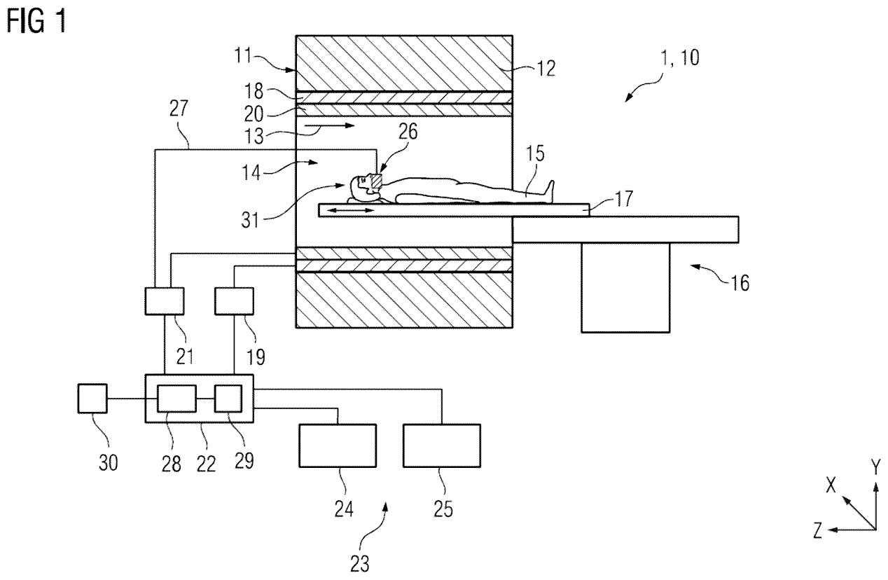

FIG. 1 shows an aspect of an imaging apparatus 1 according to the disclosure. In the present example, the imaging apparatus 1 is designed as a magnetic resonance device 10. The magnetic resonance device 10 comprises a magnet unit 11 which has e.g., a permanent magnet, an electromagnet or a superconducting main magnet 12 for generating a strong and in particular homogeneous main magnetic field 13 (B0 magnetic field). The magnetic resonance device 10 additionally comprises a patient receiving zone 14 for accommodating a patient 15. In the present exemplary aspect, the patient receiving zone 14 is designed in the shape of a cylinder and is surrounded in a circumferential direction by the magnet unit 11. Basically, however, aspects of the patient receiving zone 14 differing from this example are also conceivable.

The patient 15 can be positioned in the patient receiving zone 14 by means of a patient support and positioning device 16 of the magnetic resonance device 10. For this purpose the patient support and positioning device 16 has a patient table 17 designed to be movable inside the patient receiving zone 14.

The magnetic resonance device 10 additionally comprises a gradient coil 18 for generating magnetic gradient fields, which is used for spatial encoding during a magnetic resonance measurement. The gradient coil 18 is driven by means of a gradient control unit 19 of the magnetic resonance device 10.

The magnetic resonance device 10 can further comprise a radiofrequency antenna, which in the present exemplary aspect is designed as a body coil 20 permanently integrated in the magnetic resonance device 10. The bodycoil 20 is configured for exciting nuclear spins, which are located in the main magnetic field 13 generated by the main magnet 12. The bodycoil 20 is driven by a radiofrequency unit 21 of the magnetic resonance device 10 and radiates radiofrequency excitation pulses into an image acquisition region, which is substantially formed by the patient receiving zone 14 of the magnetic resonance device 10. The bodycoil 20 can be implemented as a receive unit of the magnetic resonance device 10, which is designed to receive magnetic resonance signals from the patient receiving zone 14.

The magnetic resonance device 10 comprises a control unit 22 for controlling the main magnet 12, the gradient control unit 19, and the radiofrequency unit 21. The control unit 22 is designed to control an execution of an imaging sequence of the imaging examination, such as e.g., a GRE (gradient echo) sequence, a TSE (turbo spin echo) sequence or a UTE (ultra-short echo time) sequence. In addition, the control unit 22 comprises a computing unit 28 for evaluating magnetic resonance signals that are captured during a magnetic resonance examination.

Furthermore, the magnetic resonance device 10 comprises a user interface 23, which has a signal connection to the control unit 22. Control information, such as e.g., imaging parameters of the magnetic resonance examination, but also reconstructed image data of a body region 31 of the patient 15, as well as an aid for adjusting an imaging parameter, can be displayed for a user on an output unit 24 of the user interface 23. For this purpose, the output unit 24 can comprise one or more monitors, for example. The output unit 24 can be configured in particular for providing a graphical user interface with image data of the body region 31 of the patient and the aid for adjusting the imaging parameter. Preferably, the user interface 23 has an input unit 25 which enables the user to adjust imaging parameters, in particular a parameter of an imaging region. The input unit 25 can be designed to enable the user to adjust a dimension, an orientation, and/or a position of a graphical object which represents the imaging region as a function of the image data of the body region 31 of the patient 15 and the aid for adjusting the imaging region.

In the present example, the computing unit 28 is connected to a memory unit 29 of the magnetic resonance device 10 by means of a signal connection. Optionally, the computing unit 28 can also be connected to a cloud 30 by means of a signal connection. The computing unit 28 can be designed to store data such as patient information, image data, localizer image data, magnetic resonance images, X-ray images or the like, in the memory unit 29 and/or the cloud 30 and/or to retrieve corresponding data from the memory unit 29 and/or the cloud 30 by means of a suitable interface (not shown). It is conceivable that the computing unit 28 is designed to obtain patient information of the patient 15 from the cloud 30 and/or the memory unit 29. It is also conceivable that the computing unit 28 is designed to obtain information about the body region 31 of the patient 15 as a function of patient information, in particular a name and/or some other form of identification, from the cloud 30 and/or the memory unit 29.

In a preferred aspect, the magnetic resonance device 10 comprises a dental coil 26 which is positioned in an appropriate position for use on the jaw region 31 and/or in an oral cavity of the patient 15. The dental coil 26 can have an antenna element (not shown) which is designed to capture magnetic resonance signals of the jaw region and/or the tooth region of the patient 15 and to transmit said signals to the computing unit 28 and/or the control unit 22.

In the present example, the dental coil 26 comprises an electric connecting cable 27, which provides a signal connection to the radiofrequency unit 21. In a preferred aspect, the dental coil 26 is designed to excite nuclear spins in the jaw region 31 of the patient 15. For this purpose, the dental coil 26 can be driven by the radiofrequency unit 21. In one example, the dental coil 26 is implemented as a mask which is positioned in an appropriate position for use on a skin surface of the jaw region 31 of the patient 15. However, it is also conceivable that the dental coil 26 is mechanically connected to a bite element which is positioned in an appropriate position for use on a dental arch, in particular along an occlusal plane, of the patient 15.

The magnetic resonance device 10 according to the disclosure may, of course, comprise further components which are typically included in magnetic resonance devices. It is also conceivable that instead of the cylindrical design, the magnetic resonance device 10 has magnetic-field-generating components arranged in a C-shaped, triangular, or asymmetric configuration. The magnetic resonance device 10 can be designed in particular to perform a magnetic resonance examination of a standing or sitting patient 15.

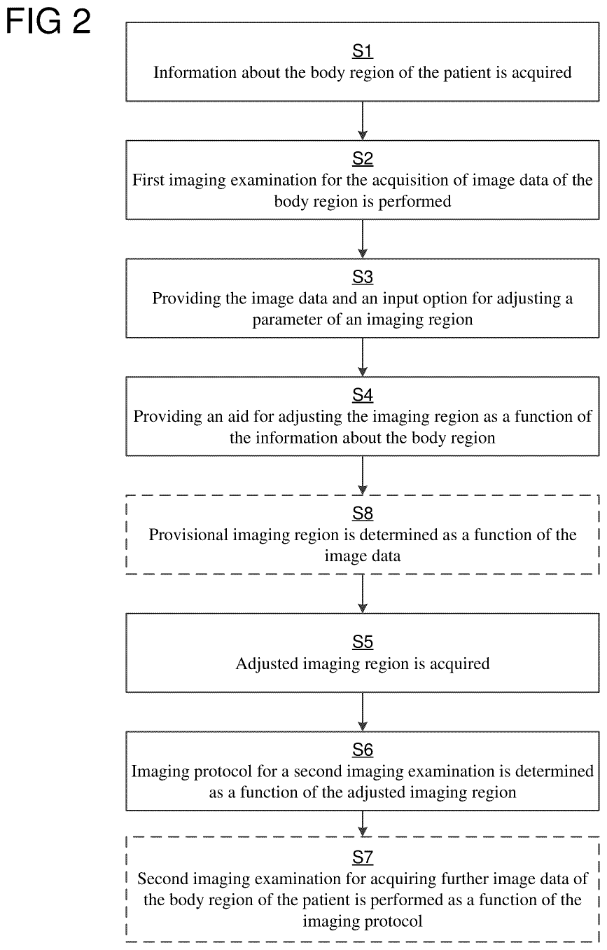

FIG. 2 shows a flowchart of an aspect of a method according to the disclosure for determining an imaging protocol for the acquisition of image data of a body region of a patient by means of an imaging apparatus 1. The workflow sequence of the exemplary aspect of the method is explained below with reference to a magnetic resonance device 10. It goes without saying that the method according to the disclosure can also be performed by means of a different imaging apparatus according to an above-described aspect.

In step S1, information about the body region of the patient is acquired. It is conceivable that the information about the body region of the patient is input by a user of the magnetic resonance device 10 by means of the user interface 23. For this purpose, the user interface 23 can comprise any desired input unit 25 that supports an interaction between the user and a graphical user interface. Preferably, the input unit 25 comprises a keyboard, a mouse, and/or a touchscreen. The information input by the user about the body region of the patient 15 can subsequently be acquired and processed by means of the control unit 22 and/or the computing unit 28 of the magnetic resonance device 10.

It is also conceivable that the information about the body region of the patient is acquired or retrieved as a function of patient information from a radiological information system, a hospital information system, a network storage unit, a local memory unit 29, and/or a cloud 30.

In step S2, a first imaging examination for the acquisition of image data of the body region is performed as a function of the information about the body region.

The control unit 22 and/or the computing unit 28 are/is preferably designed to determine, as a function of the information about the body region of the patient 15, a first imaging protocol or a first imaging region that characterizes the first imaging examination. The magnetic resonance device 10 can, in particular, be designed to perform the first imaging examination as a function of the first imaging region and/or the first imaging protocol. For example, the first imaging protocol and/or the first imaging region can be selected automatically as a function of the information about the body region by means of the control unit 22 and/or the computing unit 28 or be specified by a user of the magnetic resonance device 10 by means of the user interface 23. It is furthermore conceivable that the first imaging protocol and/or the first imaging region represent a part of the information about the body region of the patient 15.

The first imaging examination can be designed to acquire low-quality, low-resolution magnetic resonance data or image data of the body region of the patient. In particular, the first imaging examination can constitute a localizer measurement. It is also conceivable that the first imaging examination comprises an imaging examination or magnetic resonance examination which prioritizes a speed of acquisition of image data over the quality and/or a resolution of the image data.

Step S3 comprises providing the image data and an input option for adjusting a parameter of an imaging region. Preferably, the image data of the body region of the patient acquired in step S2 is provided to the user of the magnetic resonance device 10 by means of the output unit 25. The providing of the image data can, in particular, comprise displaying the image data on a monitor of the user interface 23.

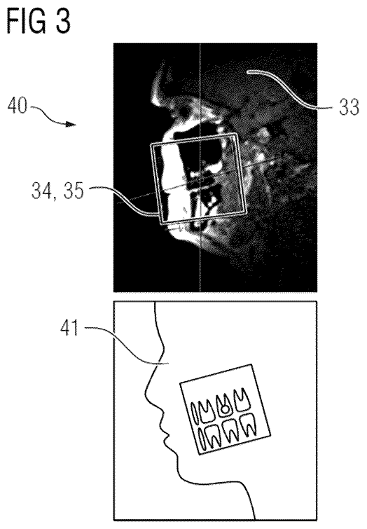

The provided image data of the body region of the patient and the input option for adjusting the parameter of the imaging region preferably constitute a part of an output 40 of the user interface 23 (see FIGS. 3 to 5) which enables the user to adjust a magnetic resonance examination, in particular a parameter of an imaging region for a further imaging examination.

One input option for adjusting the parameter of the imaging region may comprise, for example, a text-based input mask and/or a text-based input window. It is, however, also conceivable that the input option for adjusting the parameter of the imaging region comprises a graphical representation of the imaging region or a graphical object that can be adjusted by the user. For example, the user can adjust a dimension, an orientation, and/or a position of the representation of the imaging region in order to adjust the parameter of the imaging region. The control unit 22 and/or the computing unit 28 of the magnetic resonance device 10 are preferably designed to derive or determine the adjustment of the parameter on the basis of the representation of the imaging region changed by the user.

Step S4 of the method, according to the disclosure, comprises providing an aid for adjusting the imaging region as a function of the information about the body region.

In a preferred aspect, providing the aid for adjusting the imaging region comprises an outputting of a text-based user guide and/or a graphical user guide. The graphical user guide may, in particular, comprise a representation of the body region 31 of the patient 15 and of the imaging region (see FIGS. 3 to 5). The aid for adjusting the imaging region can enable a user of the magnetic resonance device 10 to adjust the imaging region for a second imaging examination.

The aid for adjusting the imaging region is preferably provided as a graphical user guide synchronously in time with a representation of the provisional imaging region and the image data of the body region 31 of the patient 15. This enables the user to adjust the imaging region as a function of a difference between the provisional imaging region, the aid for adjusting the imaging region, but also a shape of an individual anatomical structure of the body region 31 of the patient 15.

In the optional step S8, a provisional imaging region is determined as a function of the image data, wherein the providing of the input option for adjusting the parameter of the imaging region comprises superimposing the image data of the body region 31 of the patient 15 as an overlay on a representation of the provisional imaging region.

The provisional imaging region is preferably determined automatically by means of the control unit 22 and/or the computing unit 28 of the magnetic resonance device 10 as a function of the image data of the body region 31 of the patient 15. It is conceivable that the control unit 22 and/or the computing unit 28 comprise an image processing algorithm which is designed to identify the body region 31 or a diagnostically relevant section of the body region 31 of the patient 15 in the image data and to determine and/or provide the provisional imaging region adjusted to match the body region 31 or the diagnostically relevant section of the body region 31 of the patient 15.

In a preferred aspect, the provisional imaging region comprises a representation of an imaging region, in particular a graphical object which is arranged superimposed on the image data in an appropriate manner for use relative to the body region 31 of the patient 15. The graphical object can indicate a position, an orientation and/or a dimension of the imaging region which can be changed interactively by the user by means of the user interface 23 during the adjustment of the imaging region.

In a further step S5, an adjusted imaging region is acquired. Preferably, an imaging region adjusted by the user by means of the user interface 23 is acquired by the control unit 22 and/or the computing unit 28. For example, the control unit 22 and/or the computing unit 28 can be designed to acquire a position, dimension and/or orientation of a provisional imaging region changed by the user and/or a graphical object which represents the imaging region and to derive the adjusted imaging region therefrom. It is conceivable that the acquisition of the adjusted imaging region comprises a registering of an input by a mouse, a keyboard and/or a touch input of a touchscreen.

The acquisition of the adjusted imaging region can in particular comprise a storing of the adjusted imaging region in the memory unit 29, in a cloud storage 30, in a network storage or the like.

In a step S6, the imaging protocol for a second imaging examination is determined as a function of the adjusted imaging region. The imaging protocol for the second imaging examination can in particular be determined automatically on the basis of the adjusted imaging region.

The imaging protocol for the second imaging examination is preferably determined automatically by means of the control unit 22 and/or the computing unit. It is conceivable that the computing unit 28 comprises an algorithm which is designed to determine the imaging protocol for the second imaging examination as a function of the adjusted imaging region. For example, the algorithm can be designed to detect a change to a dimension, a position and/or an orientation of the provisional imaging region made by the user and to translate this into an imaging protocol for the second imaging examination. However, the control unit 22 and/or the computing unit 28 may also be designed to determine the imaging protocol on the basis of numerical data and/or of a text-based input by the user.

In an optional step S7, a second imaging examination for acquiring further image data of the body region 31 of the patient 15 is performed as a function of the imaging protocol. The second imaging examination is preferably performed by means of the imaging apparatus 1, in particular the magnetic resonance device 10.

In order to perform the second imaging examination, the patient 15 can be positioned in an appropriate position for use, as shown in FIG. 1, in the patient receiving zone 14 of the magnetic resonance device 10. The second imaging examination is, for example, a magnetic resonance examination of a tooth region or a section of a tooth region of the patient 15. Preferably, a resolution of further image data acquired by means of the second imaging examination is higher than a resolution of the image data. It is also conceivable that a diagnostically relevant section of the body region 31 of the patient 15 is more accurately centered and/or focused in the further image data than in the image data of the first imaging examination.

FIG. 3 shows a schematic representation of an aid 41 for adjusting the imaging region according to an aspect of the method. The exemplary depiction in FIG. 3 can, in particular, represent an output 40 of the output unit 25 and/or the user interface 23 for a user of the imaging apparatus 1.

In the present example, providing the image data 33 and the input option 34 for adjusting the parameter of the imaging region 35 according to step S3 comprises a juxtaposition of the image data 33 of the patient 15 with the aid 41 for adjusting the imaging region. The aid 41 for adjusting the imaging region comprises in particular a graphical user guide containing a representation of the body region 31 of the patient 15 and a representation of a desired or ideal imaging region.

Preferably, a representation of the provisional imaging region 35 is shown superimposed as an overlay on the image data 33 of the body region 31 of the patient 15. The representation of the provisional imaging region 35 can simultaneously constitute a graphical object which enables the user to adjust the imaging region 35. The provisional imaging region 35 can be proposed automatically to the user as a function of the information about the body region 31 and be based on a desired or ideal imaging region for a specific body region or a specific section of a body region. For example, the control unit 22 and/or the computing unit 28 can be designed to select or determine the provisional imaging region 35 as a function of the information about the body region 31 of the patient 15 and/or of the image data of the body region 31 of the patient 15.

In the present example, providing the image data 33 and the input option for adjusting the parameter of the imaging region 35 comprises an outputting of a sectional view of the body region 31 of the patient 15 based on the image data 33. In the present case, both the image data 33 of the body region 31 of the patient 15 and the graphical user guide (of the aid for adjusting the imaging region 35) each show a sectional view of the head or the tooth region of the patient 15. In the example shown, the reference planes of the sectional views of the image data 33 of the body region 31 of the patient 15 as well as the graphical user guide are aligned parallel to a sagittal plane of the patient 15.

Preferably, the input option 34 for adjusting the parameter of the imaging region 35 or the provisional imaging region 35 comprises a graphical object which permits the user to adjust the imaging region 35 as a function of the aid 41 for adjusting the imaging region by manipulation of a dimension, a position and/or an orientation of the graphical object.

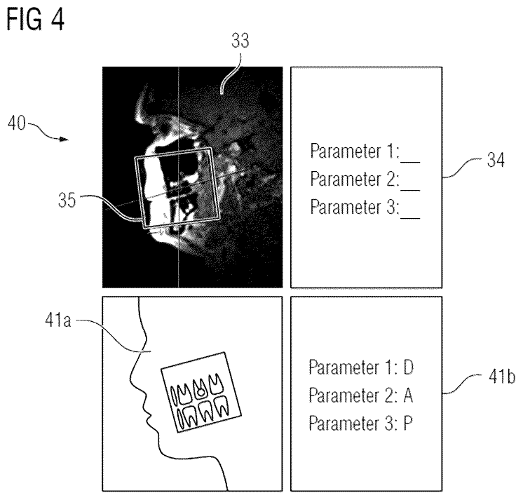

FIG. 4 shows a schematic representation of an aid 41 for adjusting the imaging region analogously according to a further aspect of the method. The exemplary depiction in FIG. 3 can in particular represent an output 40 of the output unit 25 and/or of the user interface 23 for a user of the imaging apparatus 1.

In contrast to the aspect shown in FIG. 3, the input option 34 for adjusting the parameter of the imaging region 35 comprises a text field for inputting parameters of the imaging region 35. For example, parameter 1 can define a dimension D, parameter 2 an orientation A and parameter 3 a position of the imaging region 35. At the same time, it is also possible to input further parameters that are relevant to the respective imaging apparatus 1. It is further conceivable that the input option 34 for adjusting the parameter of the imaging region 35, as shown in FIG. 3, comprises a graphical object that allows the user to make a graphical adjustment of the imaging region 35 according to the above-described aspect. Parameters 1 to 3 are preferably updated by the user as a function of a manipulation of the graphical object.

In the example shown in FIG. 4, the aid 41 for adjusting the imaging region 35 comprises both a graphical user guide 41a and a text-based user guide 41b. The text-based user guide 41b can comprise a desired or ideal parameterization of the imaging region 35 for a specific body region or a specific section of a body region of the patient 35. In the present example, the text-based user guide 41b provides the user with a desired dimension D, a desired orientation A, and a desired position P of the imaging region 35 for a left section of an upper dental arch of the patient 15. This accordingly enables the user to adjust the imaging region 35 as a function of the aid 41 for adjusting the imaging region 35 and the image data 33 containing the actual shape of the anatomical structures of the patient 15.

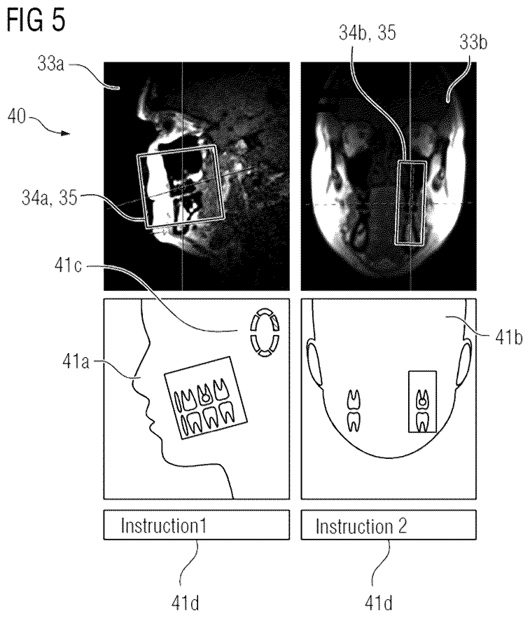

FIG. 5 shows a further schematic representation of an aid 41 for adjusting the imaging region according to a preferred aspect of the method. Analogously to the aspects shown in FIGS. 3 and 4, the image data 33 of the body region 31 of the patient 15 and the input option 34 for adjusting the parameter of the imaging region 35 are juxtaposed with a graphical user guide for adjusting the imaging region.

In the example of FIG. 5, providing the aid 41 for adjusting the imaging region 35 comprises an outputting of a graphical user guide containing representations of the body region 31 of the patient 15 and a desired or ideal imaging region. In the present case, the graphical user guide comprises two sectional views 41a and 41b of the body region 31 of the patient 15, wherein the two sectional views 41a and 41b are aligned along different reference planes. In the example shown, the reference plane of sectional view 41a is aligned parallel to a sagittal plane of the patient 15, while the reference plane of sectional view 41b is aligned parallel to a frontal plane of the patient 15.

Preferably, providing the image data 33 and the input option 34 for adjusting the parameter of the imaging region 35 further comprises an outputting of two sectional views 33a and 33b of the body region of the patient based on the image data, wherein a reference plane of each of the two sectional views 33a and 33b of the body region 31 of the patient 15 based on the image data is aligned parallel to a reference plane of each of the two sectional views 41a and 41b of the graphical user guide.

The sectional views 33a and 33b of the body region 31 of the patient 15, based on the image data, each contain an input option 34a and 34b for adjusting the parameter of the imaging region 35. In the present example, the input options 34a and 34b are designed as graphical objects which can be manipulated by the user according to an above-described aspect. A dimension, an orientation, and/or a position of the graphical objects of input options 34a and 34b for adjusting the parameter of the imaging region 35 are preferably updated if the user has performed an adjustment of the imaging region 35.

Since a left-right positioning of the imaging region 35 relative to the tooth region of the patient 15 may be unclear solely from the sectional view 41a, the sectional view 41a preferably comprises an overview map 41c of the body region 31 of the patient 15 in which one or more diagnostically relevant sections of the tooth region of the patient 15 are marked or highlighted. The overview map 41c can be generated automatically by means of the control unit 22 and/or the computing unit 28 and output together with the aid 41 for adjusting the imaging region 35. It is conceivable that the aids 41 for adjusting the imaging region of the aspects shown in FIGS. 3 and 4 also comprise a corresponding overview map 41c.

It is conceivable that the overview map 41c is provided to the user as an input option in step S1 in order to acquire the information about the body region 31 of the patient 15.

In an aspect of the method according to the disclosure, providing the aid 41 for adjusting the imaging region as a function of the body region 31 of the patient 15 further comprises an outputting of a text-based user guide. In the example shown in FIG. 5, the text-based user guide contains two instructions 41d, which comprise instructions relating to the workflow sequence of the adjustment of the imaging region 35. For example, instruction 1 instructs the user to position the imaging region 35 over a diagnostically relevant tooth group, which is to be scanned by means of the second imaging examination. Instruction 2 can instruct the user to align the imaging region 35 relative to a diagnostically relevant section of the tooth group.

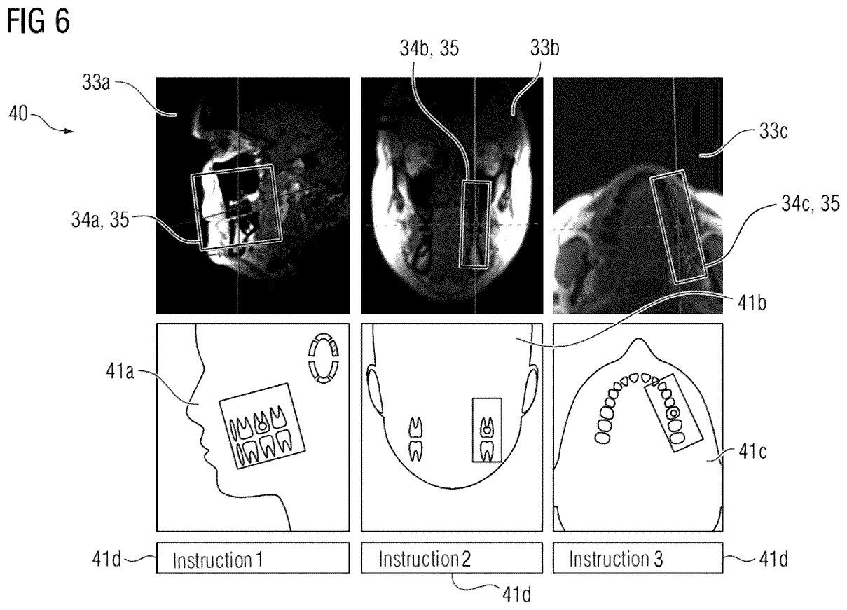

FIG. 6 shows a further schematic representation of an aid 41 for adjusting the imaging region according to a preferred aspect of the method according to the disclosure. Analogously to the aspects shown in FIG. 5, the image data 33 of the body region 31 of the patient 15 and the input option 34 for adjusting the parameter of the imaging region 35 are juxtaposed with a graphical user guide for adjusting the imaging region.

In the example shown in FIG. 6, providing the aid 41 for adjusting the imaging region comprises an outputting of a graphical user guide containing representations of the body region 31 of the patient 15 and a desired or ideal imaging region. In the present example, the graphical user guide comprises three sectional views 41a, 41b, and 41c of the body region 31 of the patient 15, which are aligned along different reference planes. In the example shown, the reference plane of sectional view 41a is aligned parallel to a sagittal plane of the patient 15. The reference plane of sectional view 41b is aligned parallel to a frontal plane of the patient 15 and the reference plane of the sectional view 41c is aligned parallel to a transverse plane or occlusal plane of the patient.

Preferably, the providing of the image data 33 and the input option 34 for adjusting the parameter of the imaging region 35 further comprises an outputting of three sectional views 33a, 33b and 33c of the body region 31 of the patient 15 based on the image data, wherein a reference plane of each of the three sectional views 33a, 33b and 33c of the body region of the patient based on the image data 33 is aligned parallel to a reference plane of each of the three sectional views 41a, 41b and 41c of the graphical user guide.

The sectional views 33a, 33b, and 33c of the body region of the patient, based on the image data, each contain an input option 34a, 34b, and 34c for adjusting a parameter of the imaging region 35. In the present example, the input options 34a, 34b, and 34c are designed as graphical objects that can be manipulated by the user according to an above-described aspect. A dimension, an orientation, and/or a position of the graphical objects of the input options 34a, 34b, and 34c are preferably updated if the user performs a corresponding adjustment of the imaging region 35.

Providing the aid 41 for adjusting the imaging region 35 can further comprise an outputting of one or more text-based user guides 41d, which, for example, contain instructions relating to the workflow sequence of the adjustment of the imaging region 35. The text-based user guides 41d can inform the user with regard to boundary conditions to be observed during the adjustment of the imaging region 35 by means of the input options 34a, 34b, and 34c. For example, the text-based user guides 41d can inform the user about an advantageous order of the adjustment of the imaging region 35 by means of the input options 34a, 34b and 34c in the sectional views 33a, 33b and 33c of the body region 31 of the patient 15 based on the image data. In particular, the text-based user guides 41d can provide the user with information about which parameter of the imaging region 35 is adjusted in relation to which sectional view 33, based on the image data, in order to achieve a time-efficient method workflow and/or to avoid errors when adjusting the imaging region 35.

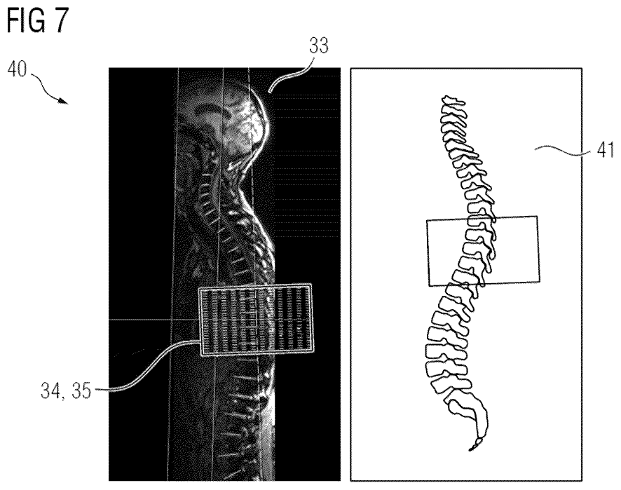

In FIGS. 3 to 6, the body region 31 of the patient 15 is a jaw region and/or a tooth region of the patient 15. FIG. 7 shows an aspect of the method according to the disclosure in which the providing of the image data 33 of the body region 31 of the patient 15 and the aid 41 for adjusting the imaging region 35 relate to the spinal column or a section of the spinal column of the patient 15. Analogously to the above-described aspects, the image data 33 of the spinal column of the patient 15 is juxtaposed with a graphical user guide 41 for adjusting the imaging region 35. In this case, a representation of the provisional imaging region 35 is shown superimposed as an overlay on the image data 33 of the spinal column of the patient 15. The representation of the provisional imaging region 35 can simultaneously comprise a graphical object 34, which enables the user to adjust the imaging region 35.

The method according to the disclosure can, of course, be used for determining an imaging protocol for the acquisition of image data of any other body regions. In particular, the method according to the disclosure for determining an imaging protocol for the acquisition of image data of body regions can be used with a plurality of differently oriented sections or subregions, such as e.g., a hand, a foot, a tooth region, but also other anatomical structures and/or organs.

The aspects of the method, according to the disclosure described herein, and the imaging apparatus, according to the disclosure, are to be understood as by way of example. If not explained in detail otherwise, individual aspects can generally be expanded by features of other aspects. In particular, the order of the method steps of the method according to the disclosure is to be understood as by way of example. The individual steps can also be performed in a different order or partially or completely overlap one another with respect to time. For example, the steps of the acquisition of the information about the body region and of the performance of the first imaging examination can be executed one after the other in a random order or at least partially overlapping. Similarly, the providing of the image data and of the input option for adjusting the parameter of the imaging region as well as the providing of the aid for adjusting the imaging region as a function of the information about the body region can be performed in any desired order one after the other or at least partially overlapping.

1. A computer-implemented method for determining an imaging protocol for acquisition of image data of a body region of a patient using an imaging apparatus, the method comprising:
acquiring information about the body region;
performing a first imaging examination as a function of the information about the body region and acquiring image data of the body region;
providing the image data and an input option for adjusting a parameter of an imaging region;
providing an aid for adjusting the imaging region as a function of the information about the body region;
acquiring an adjusted imaging region; and
determining the imaging protocol for a second imaging examination as a function of the adjusted imaging region.

2. The method as claimed in claim 1, further comprising:
determining a provisional imaging region as a function of the image data,
wherein providing the input option for adjusting the parameter of the imaging region comprises superimposing the image data of the body region of the patient as an overlay on a representation of the provisional imaging region.

3. The method as claimed in claim 2, wherein the acquisition of the adjusted imaging region comprises acquiring a correction of the provisional imaging region.

4. The method as claimed in claim 1, further comprising:
performing the second imaging examination for acquiring further image data of the body region of the patient as a function of the imaging protocol.

5. The method as claimed in claim 1, wherein providing the aid for adjusting the imaging region comprises an outputting of a text-based user guide and/or a graphical user guide.

6. The method as claimed in claim 5, wherein providing the aid for adjusting the imaging region comprises the outputting of a graphical user guide, and wherein the graphical user guide comprises a representation of the body region and of the imaging region.

7. The method as claimed in claim 6, wherein the graphical user guide comprises at least two sectional views of the body region of the patient, and wherein the at least two sectional views of the body region of the patient are aligned along different reference planes that are preferably aligned orthogonally to one another.

8. The method as claimed in claim 7, wherein providing the image data and the input option for adjusting the parameter of the imaging region comprises an outputting of at least two sectional views of the body region of the patient based on the image data, and wherein a reference plane of each of the at least two sectional views of the body region of the patient based on the image data is aligned parallel to a reference plane of each of the at least two sectional views of the body region of the patient of the graphical user guide.

9. The method as claimed in claim 6, wherein providing the image data and the input option for adjusting the parameter of the imaging region comprises juxtaposing the image data with the graphical user guide.

10. The method as claimed in claim 1, wherein the body region comprises an anatomical region of a first half of the body of the patient, which is formed substantially symmetrically to an anatomical region in a second half of the body of the patient opposite the first half of the body.

11. The method as claimed in claim 1, wherein the body region of the patient comprises a jaw region and/or a tooth region.

12. The method as claimed in claim 7, wherein a reference plane of a first sectional view of the at least two sectional views of the body region of the patient is aligned parallel to an occlusal plane of the patient along a section of a dental arch, and a reference plane of a second sectional view of the at least two sectional views of the body region of the patient is aligned orthogonally to the reference plane of the first sectional view of the body region of the patient.

13. An imaging apparatus for acquisition of image data of a body region of a patient, the apparatus comprising:
a control unit;
an output unit; and
a user interface,
wherein the control unit is designed to coordinate a method as claimed in claim 1 and to perform the method using the imaging apparatus, wherein the output unit is designed to provide a user of the imaging apparatus with the aid for adjusting the imaging region, and wherein the user interface is designed to enable the user to adjust the imaging region as a function of the aid for adjusting the imaging region.

14. The imaging apparatus as claimed in claim 13, wherein the imaging apparatus is embodied as a magnetic resonance device.

15. A non-transitory computer program product which can be loaded directly into a memory unit of a computing unit of an imaging apparatus for acquisition of image data of a body region of a patient, the apparatus including a control unit, an output unit, and a user interface, wherein the output unit is designed to provide a user of the imaging apparatus with aid for adjusting the imaging region, and wherein the user interface is designed to enable the user to adjust the imaging region as a function of the aid for adjusting the imaging region, wherein the non-transitory computer program product has program code for performing a computer-implemented method as claimed in claim 1 when the computer program product is executed in the computing unit of the imaging apparatus.
